(19) United States
(12) Patent Application Publication
(10) Pub. No.: US 2012/0101444 A1
(43) Pub. Date: Apr. 26, 2012

(54) Medical Delivery Device

(75) Inventors: Stephan Müller-Pathle, Frankfurt am Main (DE); Andrew Tubb, Gulldford (GB); Daniel Tyce, Rugby (GB); David Jonathan Glenton, Devon (GB)

(73) Assignee: SANOFI-AVENTIS DEUTSCHLAND GMBH, Frankfurt am Main (DE)

(21) Appl. No.: 13/129,294

(22) PCT Filed: Dec. 8, 2009
(86) PCT No.: PCT/EP2009/066646

§ 371 Date: Dec. 28, 2011

(30) Foreign Application Priority Data
Dec. 11, 2008 (EP) 08021483.6

(51) Int. Cl. A61M 5/31

(52) U.S. Cl. 604/189

(57) ABSTRACT

A medication delivery device comprises display means (15) having at least one segment, the segment comprising a first display element (1A1, 1B1, 1C1, 1D1, 1E1, 1F1, 1G1) and a second display element (1A2, 1B2, 1C2, 1D2, 1E2, 1F2, 1G2). The display means (15) comprises a first display layer (212) and a second display layer (222). The first display layer (212) is arranged over the second display layer (222), wherein one of the first display element (1A1, 1B1, 1C1, 1D1, 1E1, 1F1, 1G1) and the second display element (1A2, 1B2, 1C2, 1D2, 1E2, 1F2, 1G2) is located in the first display layer (212) and the other one of first display element (1A1, 1B1, 1C1, 1D1, 1E1, 1F1, 1G1) and the second display element (1A2, 1B2, 1C2, 1D2, 1E2, 1F2, 1G2) is located in the second display layer (222). The medication delivery device further comprises segment control means configured to selectively activate the at least one segment, the display elements (1A1, 1B1, 1C1, 1D1, 1E1, 1F1, 1G1, 1A2, 1B2, 1C2, 1D2, 1E2, 1F2, 1G2) of the segment being activated and visually representing status information or operation information, the first and second display elements (1A1, 1B1, 1C1, 1D1, 1E1, 1F1, 1G1, 1A2, 1B2, 1C2, 1D2, 1E2, 1F2, 1G2) being arranged such that the same information is indicated by both display elements at the same time.

The present invention relates to a medication delivery device comprising segment display means. The medication delivery device is suitable for administering doses of a medical product.

Document U.S. Pat. No. 5,559,528 shows redundant display characters having segments slit in parts such that when one of the parts of a segment fails, the other parts are still visible.

Document DE 2059427 shows a display having a first liquid crystal display and a second liquid crystal display (LCD) arranged beneath the first one. Both liquid crystal displays are configured to display the same character simultaneously. If a segment of the first LCD fails, the corresponding segment of the second LCD beneath this segment is still visible.

Display means of a medication delivery device may be suitable for indicating a dose value, which is the amount of the medical product to be injected. An indication error may have fatal consequence if a wrong dose of the medicament is injected.

It is an aim of the present invention to provide a medication delivery device having means to prevent that a wrong dose is set and injected.

For this aim, a medication delivery device comprises display means having at least one segment, the segment comprising a first display element and a second display element. The display means comprises a first display layer and a second display layer. The first display layer is arranged over the second display layer, wherein one of the first display element and the second display element is located in the first display layer and the other one of first display element and the second display element is located in the second display layer. Segment control means are configured to selectively activate the at least one segment. The display elements of the segment are activated and visually represent status information or operation information. The first and second display elements are arranged such that the same information is indicated by both display elements at the same time.

The same information is visible if one of the first display element and the second display element fails.

One embodiment of the medication delivery device comprises display means having a multitude of segments. Each segment comprises a first display element and at least one of the segments comprises the first display element and a second display element. Segment control means are configured to activate a group of segments selectively, the display elements of the group of segments being activated and visually representing status information or operation information. The activated group of segments comprises at least one segment having a first display element and a second display element arranged such that the same information is visible if one of the first display element and the second display element fails. One of the first display element and the second display element is located in the first display layer and the other one of first display element and the second display element is located in the second display layer.

The information is particularly indicated by alphabetic characters, numerals, symbols and/or marks. Even if the displayed alphabetic characters, numerals, symbols and/or marks representing the information may be slightly changed when one of the display elements fails, the information is still recognisable due to the arrangement of the first and the second display elements.

In a preferred embodiment the display means are configured to display a selected medication dose value, which is important information for the user.

In a preferred embodiment, the medication delivery device further comprises a detection device coupled to the segment control means. The detection device is configured to detect device related status information or operation information of the medication delivery device which can be displayed by the display means.

In one embodiment of the medication delivery device, one of the first and the second display elements of the segment is visible if the other display element of the activated segment fails. The first and the second display elements are duplicated such that there is no information loss if one of them fails.

In one embodiment the first and the second display elements are arranged so that the first and the second display elements do not overlap.

In an alternative embodiment the first and the second display elements of the segment are arranged so that the first and the second display elements overlap at least partly. In one embodiment one of the first and the second display elements of the activated segment is not visible if the first and the second display elements are activated. In one embodiment the first display element covers the second display element. If the first and the second display elements are arranged one upon the other, in one embodiment only the top display element is visually perceptible.

In one embodiment, the display means comprise segments having only one display element (e.g. located in the first display layer). These segments are designed to display further information which may be less important for the safe usage of the delivery device.

In one embodiment, the first display element and the second display element of one of the segments, which are located in different layers, are visible when the segment is activated.

In a preferred embodiment, the first display element and the second display element of one segment, which are located in different layers, are adjacent to each other and extend in the same direction. In other words, the segment is split in at least two display elements. In one embodiment the display elements are arranged substantially in parallel. In another embodiment the display elements of a segment are arranged serially such that they are aligned along a common axis.

An alternative embodiment has a multitude of segments each comprising first and second display elements. The first display elements of the segments each comprise a first and a second display element arranged in a first predefined pattern located at a first position and the second display elements of the same segments are arranged in a second predefined pattern located at a second position. The first and the second patterns are preferably similar. In this case the first and the second display elements of one of the segments are arranged on corresponding positions of the patterns. The information, particularly a character, numeral or symbol, is displayed twice.

In one embodiment, the display means comprise a first display configured to display elements of the first pattern and a second display configured to display elements of the second pattern.

In one embodiment, the segments are arranged as 7-segment indicator patterns. The segment control means are configured to selectively activate at least one group of the seven segments for representing the numeric characters zero to nine. 7-segment indicator patterns are suitable to display the medication dose value.

In a preferred embodiment, the display means comprise an LCD.

In a further preferred embodiment, the medication delivery device is a pen-type injector.

The design of the medication delivery device is not limited to a pen-type injector, which is very common as insulin delivery device. Alternative embodiments, in particular electronic medication delivery devices, have an alternative design.

Medication delivery devices are applied, where regular injections by persons without formal medical training, e.g. the patients, occur. This is common in the field of treatment of diabetes, where self-treatment enables patients to conduct effective management of their diabetes.

The device must be robust in construction, easy to use in terms of the manipulation of the parts, understanding by the user and the delivery of the required dose of medication. In particular the dose setting must be easy and unambiguous. In case of diabetes many users may have impaired vision requiring an easy to read dose setting display.

Medication delivery devices can be used to deliver liquid or gel medications, e.g. insulin, human growth hormone, heparin, in particular low molecular weight heparin, their analogues and their derivatives.

In one embodiment the medication is injected by a needle. An alternative embodiment of the medication delivery device is needle-less.

A medication delivery device which is used to inject several doses of medication usually has a removable needle unit, which is attached before injecting of a dose and detached after performing injection. An alternative embodiment of the medication delivery device is configured as jet injector, wherein a high-pressure narrow jet of the injection liquid is used to penetrate the patient's epidermis.

Other features will become apparent from the following detailed description when considered in conjunction with the accompanying drawings.

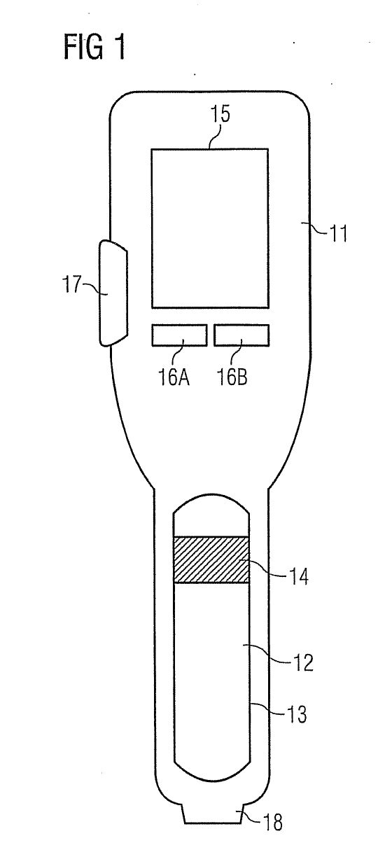

FIG. 1 shows an embodiment of a medication delivery device which is a pen type injector.

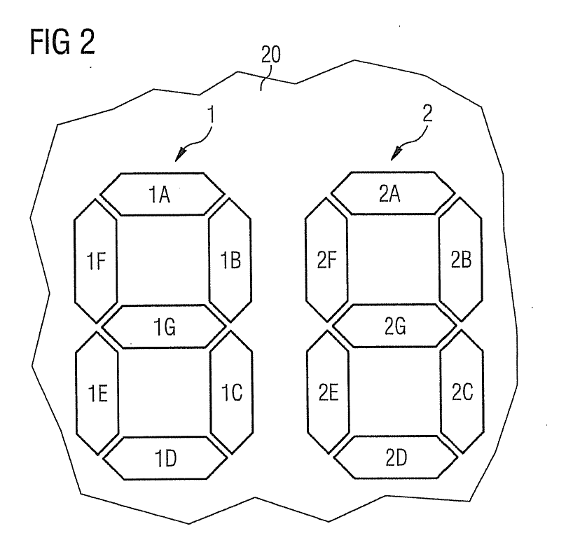

FIG. 2 shows a detailed region of an embodiment of the display means comprising two state of the art 7-segments indicator patterns.

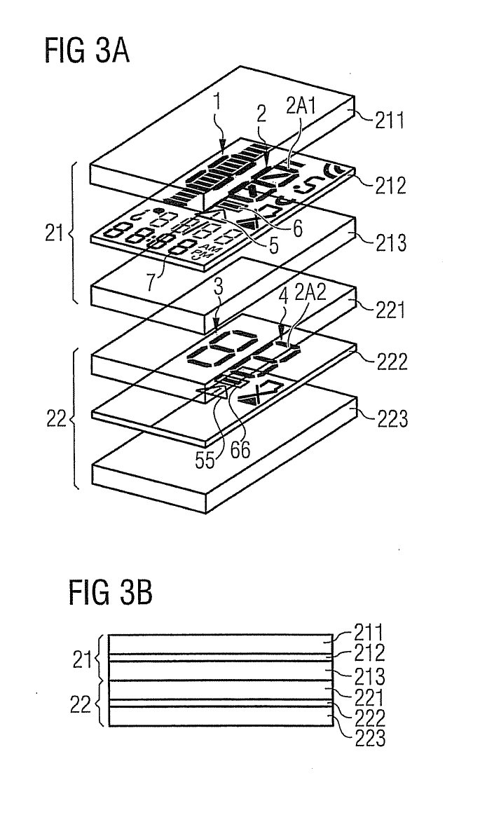

FIG. 3A shows an exploded view of an embodiment of a liquid crystal display (LCD) having two display layers.

FIG. 3B shows a cross-section of the embodiment shown in FIG. 3A.

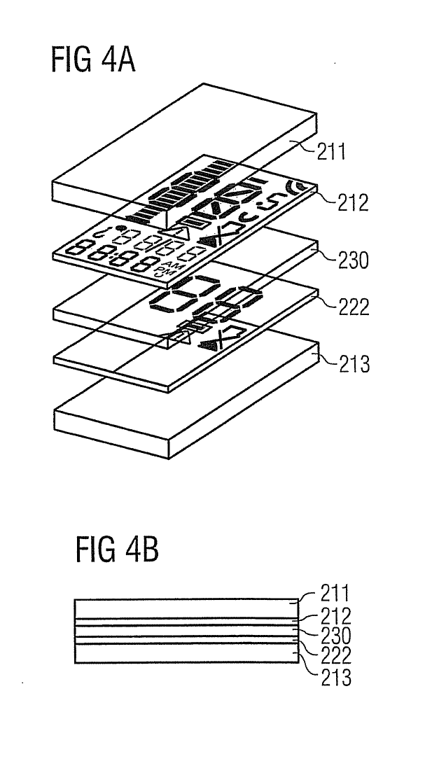

FIG. 4A shows an exploded view of an embodiment of a liquid crystal display (LCD) having two display layers.

FIG. 4B shows a cross-section of the embodiment shown in FIG. 4A.

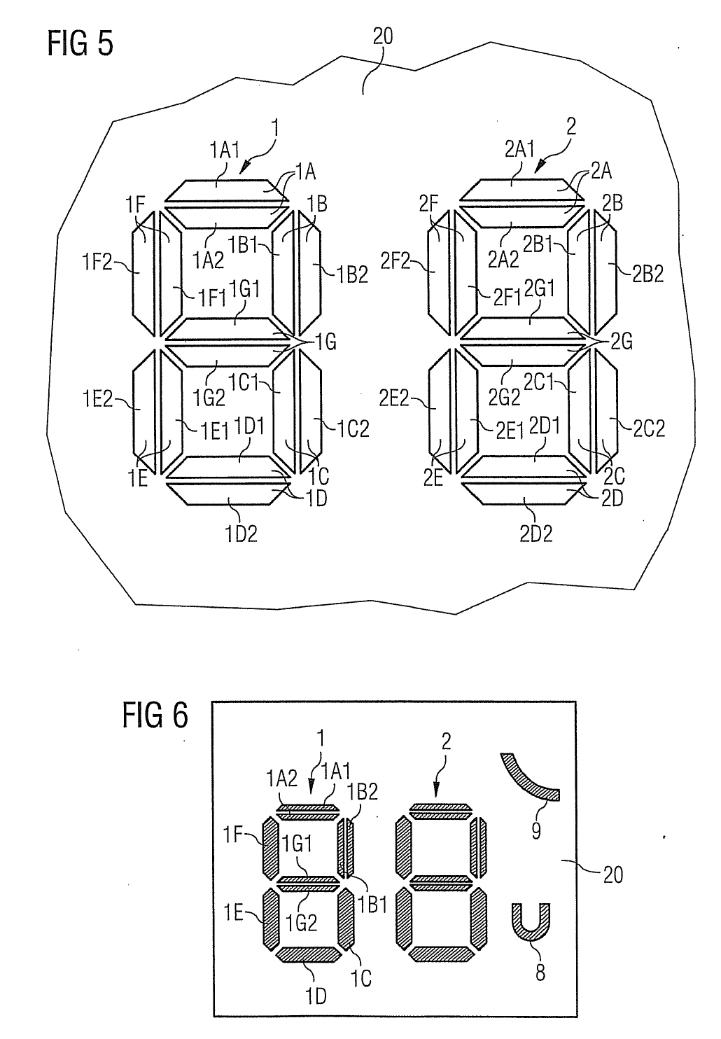

FIG. 5 shows indicators patterns which comprise a multitude of segments having display elements suitable to indicate a dose value on an embodiment of the display means.

FIG. 6 shows an embodiment of a display means having indicator patterns which comprise a multitude of segments having display elements for displaying a dose value, a unit symbol and a status indicating symbol.

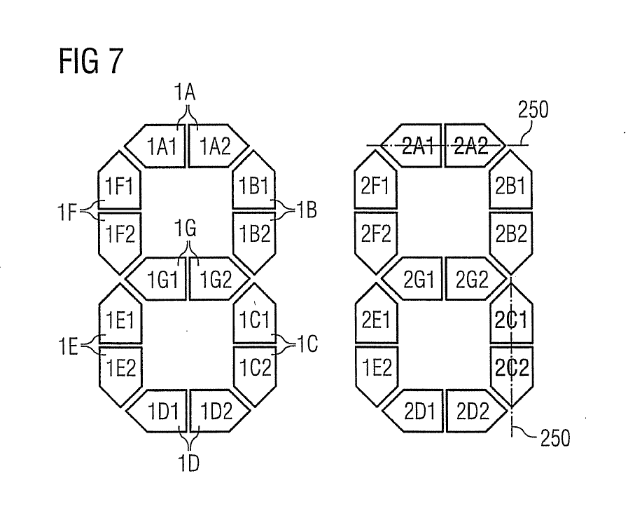

FIG. 7 shows indicator patterns which comprise a multitude of segments having display elements suitable to indicate a dose value on an embodiment of the display means.

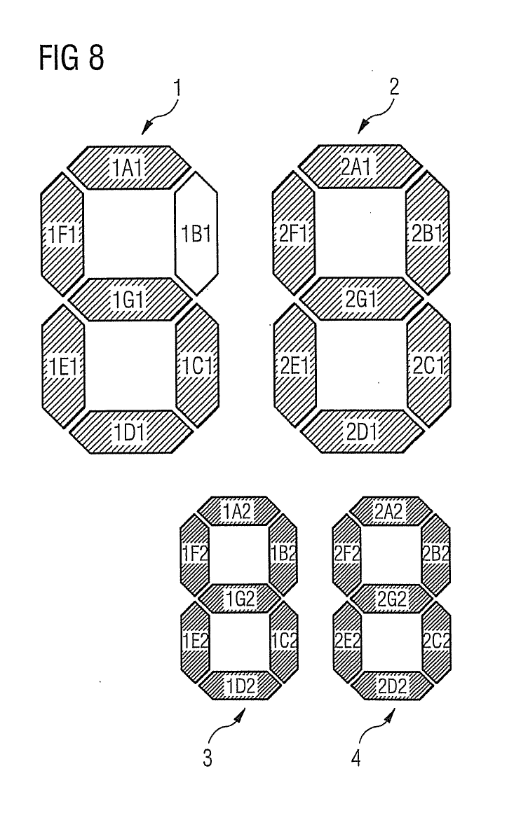

FIG. 8 shows an embodiment of redundant indicator patterns suitable to indicate a dose value on an embodiment of the display means.

FIG. 1 shows an embodiment of a medication delivery device, which is a pen type injector. The medication delivery device comprises a housing 11. The housing 11 may be designed to enable safe, correct and comfortable handling of the medication delivery device. The housing 11 may be unitary or multipart component of tubular or non-tubular shape.

The housing 11 serves to house a cartridge 12 which may be replaceable or non-replaceable. The cartridge 12, containing a liquid medical product which can be injected into the patient's body, is disposed near the distal end of the housing 11. The cartridge 12 comprises a cylinder filled with the liquid medical product and a piston 14 which is moveable inside the cylinder. The distal end of the cartridge containing part of the housing 11 is provided with a distal threaded region 18 designed for attaching a suitable needle assembly (not shown) to enable the medical product to be dispensed from the cartridge 12. When the piston 14 is moved into the distal direction, the medical product is dispensed. In this embodiment, the housing 11 comprises a window 13 through which the cartridge 12 inserted into the housing 11 is visible. The position of the piston 14 in the cylinder indicates the residual amount of the medical product in the cartridge 12.

The medication delivery device comprises electronic display means 15 arranged near the proximal end of the housing 11. The display means 15 are configured to display status information or operation information. Status information relates to the state of the medication device, e.g. charge of the battery necessary to operate the device or the amount of residual medical product in the cartridge 12. Operation information may comprise a dose value which is set or instruction information relating to setting a dose or delivering the dose for example. A detection device (not shown) located inside the housing 11 is coupled to the segment control means. The detection device is configured to detect device related status information or operation information of the medication delivery device. In one embodiment, the display means 15 comprise an LCD.

The medication delivery device comprises input means configured to adjust a dose value the patient wants to inject. In one embodiment, the input means are suitable to enter further information, e.g. time and data information. In the embodiment shown in FIG. 1, the input means are formed as buttons 16A, 16B. In an alternative embodiment the input means are designed as rotary knob (not shown). The medication delivery device further comprises a dispense button 17. In one embodiment the dispense button is arranged at the proximal end of the housing. Dispense means (not shown) located in the housing 11 are coupled to the input means, the dispense button 17 and the piston 14 such that the adjusted dose is dispensed when the dispense button 17 is pressed. One embodiment of the dispense means comprise an electromechanical device configured to read or receive the selected dose value and move the piston in the distal direction such that the selected amount of the medical product is administered when the dispense button 17 is pressed.

For setting a dose, a user enters the dose value. In one embodiment, the dose value is adjusted by the buttons 16A, 16B. Pressing one of the buttons 16A, 16B increases the dose value and pressing the other one decreases the dose value. The actually selected dose value is displayed by the display means 15. By pressing the dispense button 17, the dose is administered.

In another embodiment, the dose value is pre-set and is merely indicated by the display means for information purposes.

FIG. 2 shows a detailed region of an embodiment of the display means. The detailed region of an LCD 20 of a medication delivery device comprises two state of the art 7-segment indicator patterns 1, 2. Each indicator pattern is suitable for representing the numeric characters zero to nine.

The display comprises a first 7-segment indicator pattern 1 and a second 7-segment indicator pattern 2 each comprising seven display elements 1A, 18, 1C, 1D, 1E, 1F, 1G and 2A, 2B, 2C, 2D, 2E, 2F, 2G. Segment control means (not shown) are configured to individually activate each of the display elements 1A, 1B, 1C, 1D, 1E, 1F, 1G and 2A, 2B, 2C, 2D, 2E, 2F, 2G. They can be combined to produce simplified representations of the Arabic numerals. In one embodiment the seven display elements are arranged in an oblique or italic arrangement, which improves legibility.

Each of the numbers 0 to 9 may be represented by two or more different activated display elements 1A, 1B, 1C, 1D, 1E, 1F, 1G or 2A, 2B, 2C, 2D, 2E, 2F, 2G. The seven display elements 1A, 1B, 1C, 1D, 1E, 1F, 1G or 2A, 2B, 2C, 2D, 2E, 2F, 2G are arranged as a rectangle formed by two vertical display elements 1F, 1E; 1B, 1C or 2F, 2E; 2B, 2C on each side with one horizontal display element 1A or 2A on the top and one horizontal display element 1D or 2D on the bottom. Additionally, the seventh display element 10 or 2G bisects the rectangle horizontally. In the following, this arrangement pattern is named 7-segment indicator pattern. In further embodiments, there are also fourteen-segment displays and sixteen-segment displays suitable to display full alphanumerics.

In one embodiment of an LCD, each display element of an LCD comprises a layer of molecules aligned between two transparent electrodes and two polarizing filters. The axes of transmission of the polarizing filters are perpendicular to each other. With no liquid crystal between the polarizing filters, light passing through the first filter would be blocked by the second, crossed polarizer.

The surfaces of the electrodes are in contact with the liquid crystal material. The liquid crystal molecules are aligned in a particular direction.

The orientation of the liquid crystal molecules is determined by the alignment at the surfaces. In a twisted nematic device, the surface alignment directions at the two electrodes are perpendicular to each other, so the molecules arrange themselves in a helical structure or twist. Because the liquid crystal material is birefrigent, light passing through one polarizing filter is rotated by the liquid crystal helix as it passes through the liquid crystal layer. Thus, the light passes through the second polarized filter. Half of the incident light is absorbed by the first polarizing filter, but otherwise the entire assembly is reasonably transparent.

When a voltage is applied across the electrodes, a torque acts to align the liquid crystal molecules parallel to the electric field, distorting the helical structure. This reduces the rotation of the polarization of the incident light, and the device appears grey. If the applied voltage is large enough, the liquid crystal molecules in the centre of the layer are almost completely untwisted and the polarization of the incident light is not rotated. This light will then be mainly polarized perpendicular to the second filter and thus be blocked, and the segment will appear black.

A conventional LCD is made of two layers of clear glass or another transparent dielectric material with only one display layer in between.

Segment control means (not shown) are configured to individually activate the display elements 1A, 1B, 1C, 1D, 1E, 1F, 1G or 2A, 2B, 2C, 2D, 2E, 2F, 2G, such that a group of the display elements is visible. For example, the number “88” is displayed by activating all display elements 1A, 1B, 1C, 1D, 1E, 1F, 1G or 2A, 2B, 2C, 2D, 2E, 2F, 2G. The number “42” is displayed by activating the display elements 1F, 1G, 1B, 1C, 2A, 2B, 2G, 2E, 2D.

There is always a risk that a failure of a display element leads to an incorrect dose selection by a user who is misled by a faulty display. For example a dose value of “88”, which is displayed by activating all display elements 1A, 1B, 1C, 1D, 1E, 1F, 1G or 2A, 2B, 2C, 2D, 2E, 2F, 2G, could have a single element fault and be displayed as “68”, if the display element 1B fails. Thus, the user could receive a significant overdose.

FIG. 3A shows an exploded view of an embodiment of an LCD having two display layers. FIG. 3B shows a cross-section of the embodiment shown in FIG. 3A.

In contrast to conventional displays comprising one display layer, the embodiment shown in FIGS. 3A and 3B comprises two display layers.

The display comprises a first LCD 21 and a second LCD 22. The first LCD 21 comprises a first upper glass 211 or a sheet of another transparent dielectric material, a first lower glass 213 or a sheet of another transparent dielectric material and a first display plane 212 sandwiched between the first upper glass 211 and the first lower glass 213. The second LCD 22 comprises a second upper glass 221 or a sheet of another transparent dielectric material, a second lower glass 223 or a sheet of another transparent dielectric material and a second display plane 222 sandwiched between the second upper glass 221 and the second lower glass 223. The first LCD 21 is arranged on the second LCD 22.

The first display plane 212 is configured to display a multitude of information. It comprises a first 7-segment indicator pattern 1 and a second 7-segment indicator pattern 2 for indicating the dose value, an alarm symbol 5 and a symbol indicating low charge of battery 6. It comprises further 7-segment indicator patterns 7 for indicating time and further symbols for indicating other status and operation information.

The second display plane 222 is configured to display only information which may be considered critical for the safe usage of the medication delivery device. The second display plane 222 comprises a third 7-segment indicator pattern 3 and a fourth 7-segment indicator pattern 4 for indicating the dose value, the alarm symbol 55 and the symbol indicating low charge of battery 66.

The display elements in the second display plane 222 are duplicated for a group of the display elements in the first display plane 212. The duplicated display elements having the same shape are arranged beneath the similar display elements arranged in the first display plane 212.

Redundant elements are allocated to a same segment. For example, one of the segments comprises the alarm symbol 5 located in the first display plane 212 and the alarm symbol 55 located in the second display plane 222. Another segment comprises the top vertical display element 2A1 of the second indicator pattern 2 and the top vertical display element 2A2 of the fourth indicator pattern 4.

When a segment is activated, the display elements allocated to this segment are activated. If one of the display elements located in the first display plane is activated, the redundant display element of this display element is activated at the same time.

Segment control means (not shown) are configured to activate the segments dependent on of the information that should be indicated. “Activate” means that the translucence of the display elements assigned to the segment is reduced such that the segment seems grey or black. In case of redundant display elements being arranged one over another, e.g. the alarm symbols 5 and 55, the visible display element may be darker than a display element not having a redundant display element, because the redundant arrangement reduces translucence twice.

If an activated redundant display element located in the first display plane 212 fails, the redundant display element located in the second display plane 222 is still visually perceptible.

FIG. 4A shows an exploded view of an embodiment of an LCD having two display layers. FIG. 4B shows a cross-section of the embodiment shown in FIG. 4A.

In the following, only the difference between the embodiment shown in FIGS. 3A and 3B and the embodiment shown in FIGS. 4A and 4B are described.

The LCD comprises an upper glass 211 or a sheet of another transparent dielectric material, a first display plane 212, a transparent separation layer 230, a second display plane 222 and a lower glass 213 or a sheet of another transparent dielectric material. The first display plane 212 is sandwiched between the upper glass 211 and the separation layer 230. The second display plane 222 is sandwiched between the separation layer 230 and the lower glass 213.

Instead of two glass layers located between the first and the second display plane, as provided in the embodiment shown in FIGS. 3A and 3B, only one separation layer 230 is provided between the display planes 212, 222.

FIG. 5 shows an embodiment of two 7-segment indicator patterns 1, 2 which comprise a multitude of segments having display elements suitable to indicate a dose value on an embodiment of display means.

Each indicator pattern 1, 2 comprises 14 display elements 1A1, 1A2, 1B1, 1B2, 1C1, 1C2, 1D1, 1D2, 1E1, 1E2, 1F1, 1F2, 1G1, 1G2 and 2A1, 2A2, 2B1, 2B2, 2C1, 2C2, 2D1, 2D2, 2E1, 2E2, 2F1, 2F2, 2G1, 2G2. In each case two of the display elements are arranged as a pair of display elements extending generally parallel to each other. Each indicator pattern 1, 2 comprises seven pairs 1A, 1B, 1C, 1D, 1E, 1F, 1G and 2A, 2B, 2C, 2D, 2E, 2F, 2G of display elements arranged as a rectangle formed in each case by two vertical pairs 1F, 1E; 1B, 1C or 2F, 2E; 2B, 2C on each side with one horizontal pair 1A or 2A on the top and one horizontal pair 1D or 2D on the bottom. Additionally, in each case the seventh pair 1G or 2G bisects the rectangle horizontally.

Each indicator pattern 1, 2 comprises seven segments each comprising one pair of display elements. In one embodiment at least one of the display elements is located in a first display layer and the other display elements are located in another display layer, e.g. the first display elements are located in the first display layer and the second display elements are located in a second display layer. In one embodiment the first display element and the second display element of a segment are arranged such that they at least partly overlap. Alternatively the first display element and the second display element of the segment do not overlap.

Normally, the display elements of each one of the segments are always active or inactive at the same time at least unless a failure has caused one of the display elements to operate improperly. If one of the display elements of one of the activated segments fails, the other display element of the segment is still visually perceptible. In comparison with other activated segments comprising two visible display elements that segment looks narrower. But the dose value is still legible.

FIG. 6 shows an embodiment of an LCD 20 having two indicator patterns 1, 2 for displaying a dose value, a unit symbol 8 and a status indicating symbol 9.

Each indicator pattern 1, 2 comprises seven segments. Some of the segments comprise only one display element 1C, 1D, 1E, 1F. The other ones comprise two display elements 1A1, 1A2; 1B1, 1B2; 1G1, 1G2 arranged as pairs of display elements, the two display elements of each pair extending generally in parallel to each other. The segments of each indicator pattern 1, 2 are arranged in a 7-segment indicator pattern.

Only some of the segments of the 7-segment indicator pattern are split. Segments whose failure may cause displaying a false number are split. The following examples illustrate this aspect. The number 7 is represented by the activated segments 1A, 18, 1C (see reference numerals used in FIG. 2). If the segment 1A fails the other segments 1B and 1C represent the number 1. The number 8 is represented by the activated segments 1A, 1B, 1C, 1D, 1E, 1F, 1G. If the segment 1B fails the other segments represent the number 6. If the segment 1G fails the other segments represent the number 0.

Segment control means (not shown) are provided for activating some of the segments such that the display elements of these segments are visually perceptible. The segment control means comprise separate control lines for activating the display elements of each segment. If one of the two display elements of a segment fails or there is a failure in a corresponding control line, the other display element remains activated. Thus, a single fault would not lead to a completely missing segment.

Only the indicator patterns for displaying the dose value have redundant display elements. The dose value is the amount of the medical product to be injected. An indication error may have fatal consequence if a wrong dose of the medicament is injected. The redundant display elements should prevent injection errors possibly caused by an indication error. The unit symbol indicator 8 and the status indicator 9 do not have redundant display elements. These indicators indicate information which may not cause fatal injection errors.

FIG. 7 shows an embodiment of indicator patterns 1, 2 suitable to indicate a dose on the display means of one embodiment.

Each indicator pattern 1, 2 comprises seven pairs of display elements. Each pair comprises two display elements adjacent to each other and extending in the same direction but spaced apart. For example, one of the pairs 1A comprises the display elements 1A1 and 1A2. Another pair 1D comprises the display elements 1D1 and 1D2.

The pairs are arranged as a rectangle formed by two vertical pairs 1F, 1E; 1B, 1C on each side with one horizontal pair 1A on the top and one horizontal pair 1D on the bottom. Additionally, the seventh pair 1G bisects the rectangle horizontally.

Instead of pairs of display elements each having two display elements extending in parallel to each other, as shown in FIGS. 5 and 6, these pairs of redundant display elements each have two display elements which are serially arranged. Two display elements forming a pair are arranged end-to-end so that they define together a segment. The two display elements of each segment are arranged serially such that they are aligned along a common axis 250. The display elements 2A1 and 2A2 are aligned along a common horizontal axis 250. The display elements 2C1 and 2C2 are aligned along a common vertical axis 250, for example.

In one embodiment at least one of the display elements is located in a first display layer and the other display elements are located in another display layer, e.g. the first display elements are located in the first display layer and the second display elements are located in a second display layer. In one embodiment the first display element and the second display element of a segment are arranged such that they at least partly overlap. Alternatively the first display element and the second display elements of the segment do not overlap.

If one of the display elements of one activated segment fails the other element is still visually perceptible. In case of a failure of one of the display elements, a portion of one segment is not visible, but the displayed dose value is still recognizable.

FIG. 8 shows an embodiment of redundant segment patterns suitable to indicate a dose on a display means of one embodiment.

The display element comprises a first dose indicator pattern 1, 2 having seven display elements 1A1, 1B1, 1C1, 1D1, 1E1, 1F1, 1G1 arranged in a first 7-segment indicator pattern 1 and further seven display elements 2A1, 2B1, 2C1, 2D1, 2E1, 2F1, 2G1 arranged in a second 7-segment indicator pattern 2. A second dose indicator pattern 3, 4 has seven display elements 1A2, 1B2, 1C2, 1D2, 1E2, 1F2, 1G2 arranged in a third 7-segment indicator pattern 3 and further seven display elements 2A2, 2B2, 2C2, 2D2, 2E2, 2F2, 2G2 arranged in a fourth segment 7-segment indicator pattern 4.

A segment comprises one of the display elements of the first pattern 1 and one of the display elements of the third pattern 3. Furthermore, a segment comprises one of the display elements of the second pattern 2 and one of the display elements of the fourth pattern 4. The display elements of a segment are arranged at the same positions in their respective pattern. For example, one segment comprises display element 1A1 in the first pattern 1 and display element 1A2 in the third pattern 3. Another segment comprises display element 2E1 in the second pattern 2 and display element 2E2 in the fourth pattern 4.

In one embodiment at least one of the display elements is located in a first display layer and the other display elements are located in another display layer, e.g. the first dose indicator pattern 1, 2 is located in the first display layer and the second dose indicator pattern 3, 4 is located in a second display layer.

Segment control means (not shown) are configured for activating some or all of the segments such that the display elements of the segments are visually perceptible. In other words, the display elements arranged at the same position in the first dose indicator pattern 1, 2 and the second dose indicator pattern 3, 4 are visually perceptible. The two display elements of a segment are always active or inactive at the same time, such that the redundant number, e.g. dose value, indicated in the second dose indicator pattern is the same as the number indicated in the first dose indicator pattern.

If a failure causes one of the display elements of a segment to operate improperly, the characters displayed by the first and second dose indicator pattern differ. A patient using the display means checks whether the characters displayed by the first and second dose indicator pattern are the same. If they are not the same, the patient is able to recognize a device malfunction. The patient recognises that at least one display element fails. If they differ only by one visible display element, the correct dose value is the value having more visible display elements. For example if the first dose indicator patterns 1, 2 display “68”, as indicated by hatched display elements 1A1, 1C1, 1D1, 1E1, 1F1, 1G1, 2A1, 2B1, 2C1, 2D1, 2E1, 2F1, 2G1 and the second dose indicator patterns 3, 4 display “88”, as indicated by hatched display elements 1A2, 1C2, 1B2, 1D2, 1E2, 1F2, 1G2, 2A2, 2B2, 2C2, 2D2, 2E2, 2F2, 2G2, the user recognizes that “88” has more activated display elements and that the correct dose value is “88”. The recognition is based on the assumption that one display element of a segment fails.

In the rare case that a first display element positioned at a first position in the first dose indicator pattern 1, 2 and a second display element positioned at another position in the second dose indicator pattern 3, 4 fail, the character to be displayed is the character composed of the display elements which are visible in at least one of the first and the second dose displays. In other words, none of the first dose indicator pattern 1, 2 and the second dose indicator pattern 3, 4 shows the correct information. The user recognizes that something is wrong with the device and therefore he or she will not use it.

In this embodiment the indicated information, e.g. the dose value, is visible on a first location of the display and on a second location of the display at the same time. In an alternative embodiment the dose value is displayed on a first and a second display of the medication delivery device which are separate.

In one embodiment the dose value is displayed at least three times.

The invention is not limited to 7-segment indicator patterns, but is also suitable for any symbol. Any symbol, which indicates status indicating information and/or operation information which may be important for safe handling of the medication delivery device, can be displayed by segments having redundant display elements.

Other implementations are within the scope of the claims. Elements of different embodiments may be combined to form implementations not specifically described herein.

### REFERENCE NUMERALS

| | |
|---|---|
| 1, 2, 3, 4 | indicator patterns |
| 1A, 1B, 1C, 1D, 1E, 1F, 1G, | |
| 2A, 2B, 2C, 2D, 2E, 2F, 2G, | |
| 1A1, 1B1,1C1,1D1, 1E1, 1F1,1G1, | |
| 2A1, 2B1,2C1,2D1, 2E1, 2F1,2G1, | |
| 1A2, 1B2,1C2,1D2, 1E2, 1F2,1G2, | |
| 2A2, 2B2,2C2,2D2, 2E2, 2F2, 2G2 | display elements |
| 5, 55 | alarm symbol |
| 6, 66 | symbol indicating low |
| | charge of battery |
| 7 | indicators |
| 8 | unit symbol |
| 9 | status indicating symbol |
| 11 | housing |
| 12 | cartridge |
| 13 | window |
| 14 | piston |
| 16A, 16B | buttons |
| 17 | dispense button |
| 18 | distal threaded region |
| 20, 21, 22 | LCD |
| 211, 221 | upper glass |
| 213, 223 | lower glass |
| 212, 222 | display plane |
| 230 | separation layer |
| 250 | axis |

1. Medication delivery device comprising
a display having at least one segment, the segment comprising a first display element and a second display element, the display comprising a first display layer and a second display layer, the first display layer being arranged over the second display layer,
wherein one of the first display element and the second display element is located in the first display layer and
the other one of first display element and the second display element is located in the second display layer, and
a segment control configured to selectively activate the at least one segment, the display elements of the segment being activated and visually representing status information or operation information, the first and second display elements being arranged such that the same information is indicated by both display elements at the same time.

2. Medication delivery device according to claim 1, wherein the display comprises a plurality of segments.

3. Medication delivery device according to claim 1, wherein the display is configured to display a selected medication dose value.

4. Medication delivery device according to claim 1, further comprising
a detection device coupled to the segment control, the detection device configured to detect device related status information or operation information of the medication delivery device.

5. Medication delivery device according to claim 1, wherein the first and the second display elements are arranged such that the first and the second display elements do not overlap.

6. Medication delivery device according to claim 1, wherein the first and the second display elements are arranged such that the first and the second display elements overlap at least partly.

7. Medication delivery device according to claim 6, wherein the display element located in the first display layer covers the display element located in the second display layer.

8. Medication delivery device according to claim 1 having at least two segments, wherein at least one of the segments has only display elements located in the first display layer.

9. Medication delivery device according to claim 1, wherein the first display element and the second display element of the segment are visible when the segment is activated.

10. Medication delivery device according to claim 1, wherein the first display element and the second display element are adjacent to each other and extend in a same direction.

11. Medication delivery device according to claim 10, wherein the first display element and the second display element of the segment are arranged substantially in parallel.

12. Medication delivery device according to claim 10, wherein the first display element and the second display element of the segment are arranged serially such that they are aligned along a common axis.

13. Medication delivery device according to claim 1 having a plurality of segments each comprising first and second display elements, wherein the first display elements of the segments are arranged in a first predefined pattern located at a first position and wherein the second display elements of the same segments are arranged in a second predefined pattern located at a second position, the first and the second display elements of one of the segments being arranged at corresponding positions.

14. Medication delivery device according to claim 13, wherein the display comprises a first display device configured to display the display elements of the first pattern and a second display device configured to display the display elements of the second pattern.

15. Medication delivery device according to claim 1 having a plurality of segments, wherein the segments are arranged in a 7-segment indicator pattern, and wherein the segment control is configured to selectively activate at least one group of the seven segments for representing the numeric characters zero to nine.

16. Medication delivery device according to claim 1, wherein the display comprises an LCD.

17. Medication delivery device according to claim 1, being a pen-type injector.
